(19) United States
(12) Patent Application Publication
(10) Pub. No.: US 2020/0000918 A1
(43) Pub. Date: Jan. 2, 2020

(54) COSMETIC AND PHARMACEUTICAL AGENTS FOR TREATING WOUNDS AND/OR HEMORRHAGING OF THE SKIN, MUCOUS MEMBRANES OR THE GUM, IN PARTICULAR, FOR EXAMPLE, SUPERFICIAL CRACKS (RHAGADES) OR PERIODONTITIS

(71) Applicant: Jean-Pierre Bogaert, Monaco (MC)

(72) Inventor: Jean-Pierre Bogaert, Monaco (MC)

(21) Appl. No.: 16/458,728

(22) Filed: Jul. 1, 2019

Related U.S. Application Data
(62) Division of application No. 15/526,247, filed on May 11, 2017, now abandoned.

(30) Foreign Application Priority Data
Nov. 11, 2014 (CH) 01750/14

(51) Int. Cl. A61K 45/06; A61K 9/06; A61K 47/44; A61K 8/365; A61K 8/46; A61Q 11/00; A61K 8/73; A61Q 19/00; A61K 9/00; A61K 31/194; A61K 33/04; A61K 33/14; A61K 33/20; A61K 33/40

(52) U.S. Cl. CPC A61K 45/06; A61K 9/06; A61K 47/44; A61K 8/365; A61K 8/46; A61Q 11/00; A61K 33/40; A61Q 19/00; A61K 9/0014; A61K 31/194; A61K 33/04; A61K 33/14; A61K 33/20; A61K 8/73

(57) ABSTRACT

The invention relates to an agent, in particular a cosmetic and/or pharmaceutical agent, containing an active composition and optionally an adhesion composition, said active composition containing at least one organic or inorganic chlorine compound and at least one oxidation agent, for use in the treatment of wounds and/or hemorrhaging of the skin, mucous membranes or the gums, in particular in the absence of canker sores.

### CROSS REFERENCE TO RELATED APPLICATIONS

This application is a divisional of U.S. patent application Ser. No. 15/526,247 filed May 11, 2017, which is national phase entry under 35 U.S.C. § 371 of PCT/EP2015/076260 filed on Nov. 10, 2015, which claims priority to Swiss Patent Application No. 01750/14 filed on Nov. 11, 2014, the entirety of each of which is incorporated by this reference.

### TECHNICAL FIELD OF THE INVENTION

The invention comprises an agent, in particular a cosmetic and/or pharmaceutical agent, for treatment of wounds and/or bleeding of the skin, the mucous membranes or the gingiva, in particular for cracked skin or periodontitis.

### BACKGROUND OF THE INVENTION

In many cases today, wound healing cannot be promoted therapeutically and supported optimally. Science is still attempting to discover agents and methods for preventing complications and infections, preventing a delay in healing and optimizing the cosmetic results as much as possible. There are still patients who suffer from chronic or refractory wounds.

The Unexamined European Patent Application EP 2 777 708 A1 relates to an agent for treating aphthae or mouth ulcers, comprising a flowable pasty adhesive composition suitable for adhering to the oral mucosa after application, and an active pharmaceutical composition containing an organic or inorganic chlorine compound and at least one oxidizing agent.

U.S. Pat. No. 6,280,775 B1 discloses a mouthwash for treating and reducing dental diseases. This mouthwash is based on a mixture of a solution of a water-soluble metal chlorite and a solution containing sodium persulfate and hydrogen peroxide. The resulting composition contains chlorine dioxide.

The Unexamined Patent WO 99/34773 A1 describes antimicrobial care agent based on a peroxycarboxylic acid. This agent is supplied in particular in the form of shampoos, roller pens, gels, rinses and oral care compositions, such as mouthwashes, toothpastes, dental powders, mouth sprays, chewing gums, lozenges, sachets and bleach kits, etc., for treating topically treatable microbial infections, in particular diseases of the oral cavity, such as plaque, gingivitis, periodontitis and bad breath or anaerobic infections of the skin.

The Unexamined Patent WO 2004/000372 A2 describes a composition for disinfection of dental prostheses, toothbrushes, shaving devices and the like, containing a chloride or bromide compound in the form of a salt, at least one oxidizing agent having an oxidation potential, which is higher in solution than the oxidation potential of Cl¹⁻/Cl⁰ and/or Br¹⁻/Br⁰, and at least one acid in excess for producing a pH of <6 when the composition is completely dissolved.

The Unexamined Patent WO 00/19981 A1 discloses antimicrobial pharmaceutical preparations (e.g., solutions, gels, ointments, creams, delayed release preparations, etc.), which contain chlorite (for example, a metal salt of a chlorite) in combination with a peroxide compound (e.g., hydrogen peroxide) for use in disinfecting objects or surfaces (e.g., contact lenses, covers, etc.), antisepsis of the skin or other body parts, preventing scarring and/or treatment and prevention of diseases of the skin or mucous membranes (e.g., wounds, burns, infections, freezing, ulceration, psoriasis, acne or other scar-forming lesions). The Unexamined Patent US 2004/037891 A1 also discloses an antimicrobial preparation containing chlorite in combination with a peroxide compound. However, this composition also remains stable during storage at room temperature and does not produce any chlorine dioxide.

### ADVANTAGES

One advantage of the present invention is to make available a pain relief agent and an agent for promoting wound healing. In particular, an agent that is easy to use, such as by the patient himself, is to be made available. In particular, an agent is to be found, which contributes to pain relief and wound healing when applied topically.

In addition, another advantage of the present invention is to make available an agent (substance mixture) and a dosage form, which will permit easy, localized and long-lasting treatment.

For example, one advantage of the invention is to make available an agent for use on chapped skin or cracked skin, for example, on the soles of the feet, heels or between the toes, on the hands, fingers or between the fingers, on the lips, the corner of the mouth, the corner of the nose, in the hollows at joints or in the perianal region. In particular, one goal is to make available an agent with which it is possible to relieve and cure broken skin and/or cracked skin in particular including fissures on keratinized skin areas.

Another advantage of the present invention is to make available an agent for use in the area of the mouth or throat, in particular for problems with the gums or periodontitis.

Another advantage of the present invention is to make available an agent for topical use on injuries to the mucus membranes in the oropharyngeal area, in particular the gingiva, i.e., gums.

### SUMMARY OF THE INVENTION

These and other advantages are achieved through the features of the independent patent claims. Further embodiments and/or advantageous embodiment variants of the invention are the subject matter of the dependent patent claims.

The invention also relates to an agent, in particular a composition and/or a substance mixture containing at least one organic or inorganic chlorine compound and at least one oxidizing agent for use in the treatment of wounds and/or bleeding skin, the mucus membranes or the gingiva, in particular excluding aphtha, canker sores and thrush.

The agent according to the invention is suitable in particular for use
in the treatment of cracks in the skin, e.g., rhagades, in particular on feet and hands, in particular on the soles of the feet, the edge of the soles of the feet, the heels, toes, hand surface and/or fingers,
in treatment of non-healing wounds, including, for example, wounds on diabetic patients, in particular wounds on the extremities, e.g., legs, arms, feet, hands, toes and fingers,
in treatment of infected wounds, in particular bacterially infected wounds and/or fungus infected wounds,
in treatment of periodontitis, in particular in marginal periodontitis or as an additional treatment for apical periodontitis,
for disinfecting wounds,
in treatment of bleeding wounds, in particular, for example, in gingival bleeding and/or
in treatment of refractory (i.e., non-healing) open wounds on the skin or mucous membranes.

This agent thus makes it possible to treat even refractory chronic of non-healing refractory wounds, i.e., wounds which fail to heal and/or show no improvement for period of more than 2 days, a week, 4 weeks or more. Wound healing is accelerated in general. The agent according to the invention is thus suitable for accelerating wound healing.

In addition, the agent according to the invention acts as an anti-inflammatory or disinfectant agent, in particular when used on open areas of skin or on the gingiva. Gingival bleeding can be reduced or prevented, in particular after a dental treatment and/or in periodontitis. The agent according to the invention is suitable in particular for treatment of periodontitis, in particular marginal periodontitis or as an accessory treatment (after a dental intervention) in apical periodontitis.

The agent according to the invention also has a pain relieving or analgesic effect and may be used on the aforementioned dermal and/or mucosal defects and/or dermal and/or mucosal wounds, in particular after a dental treatment, for example, a dental cleaning treatment, or in treatment of cracked skin, in particular rhagades and thus additionally for pain relief.

The agent according to the invention is suitable in particular for use in the treatment of periodontitis in combination with a tooth cleaning procedure, in particular a scaling and root planing (SRP) treatment. The agent according to the invention is expediently used following a dental cleaning treatment, in particular following a scaling and root planing (SRP) treatment. The agent according to the invention is expediently applied to the gingiva, in particular to the gingival margins, such as by being allowed to act from 1 to 3 hours. In the days after the dental cleaning treatment or scaling and root planing (SRP) treatment (e.g., lasting at least three consecutive days, or at least five consecutive days) regular dental care may be performed additionally (e.g., at least once a day) with an aqueous solution of the agent according to the invention.

Cracked skin and/or fissures in the skin, in particular deep fissures in the skin are often the result of overstress on skin, which has a reduced elasticity, e.g., due to drying, cold (climate influences) or diseases (e.g., skin diseases) on the lips, the corners of the mouth, the sides of the nose, the soles of the feet (in particular on the heels or on or between the toes), the hands and/or fingers, the bends of joints or in the perianal region. In addition to environmental factors and diseases, cracked skin may also occur due to other causes, e.g., because of a bacterial infection in a skin injury. A fissure (i.e., a crack) in the skin, in particular a deep fissure in the skin due to overstress in areas of reduced elasticity, is also referred to medically as a rhagade and colloquially as a canker sore or ulcer. Fissures, i.e., rhagades, may also occur. These deep cracks may be unattractive as well as painful, and may in some cases be hygienically problematical and potential sites of entry for pathogens. If healing is delayed, such fissures can have a restrictive effect. Measures to promote healing are therefore desirable.

In addition, it has been found that the agent according to the invention also leads to an improvement and ultimately to healing of other types of wounds. The agent according to the invention can also be used advantageously on refractory (non-healing) wounds (e.g., there may be a negative effect on the course of healing due to circulation disorders, use of certain medications, wound infections or other reasons). Diabetic wounds in particular can be treated successfully with this agent according to the invention.

Furthermore, it has been found that bleeding gums and gingivitis in particular can be relieved rapidly.

It has been discovered that the agent according to the invention can be applied successfully by topically application.

The at least one organic or inorganic chlorine compound and the at least one oxidizing agent defined an active ingredient composition, which may optionally contain additional substances. If the active composition is combined with an adhesive composition, which is in the form of a paste, cream, ointment or gel in particular, then the treatment is a particularly long-lasting treatment and is intense due to the special adhesive properties of the agent. Any (further) penetration of microorganisms out of the environment is thereby prevented.

The agent according to the invention is also advantageously characterized in that
the active ingredient composition comprises a carboxylic acid, such as a monocarboxylic or dicarboxylic acid such as citric acid,
the acid is present in such an amount in the active composition that the active composition dissolved in an aqueous solution in a certain amount will produce a pH of <6, or <5 or <4.5,
the oxidizing agent is selected from the group comprised of persulfate compounds, in particular the oxidizing agent contains a hydrogen peroxosulfate compound as an oxidizing agent,
the chlorine compound is present in the form of an alkali or alkaline earth metal salt,
the chlorine compound comprises chloramine B (N-chlorobenzenesulfonamide sodium), chloramine T (p-toluenesulfonic chloramine sodium), dichloramine T (p-toluenesulfonic dichloramide), halazon (p-dichlorosulfamylbenzoic acid), dichlorocyanuric acid, trichlorocyanuric acid, TCM (trichloromelamine), 1,3-dichloro1,3-dichloro-5,5-dimethylhydantoin, dichloroglycoluril, succinic chlorimide or chloroazodin (N,N′-dichloro diazo dicarbonamidine) or a mixture of these substances,
the active composition contains at least potassium hydrogen monopersulfate (KHSO₅) as an oxidizing agent and a salt form of a chloride compound, such as from the group of alkali salts, e.g., table salt (NaCl),
the oxidation potential of the oxidizing unit in aqueous solution is higher at least in the claimed pH range than the oxidation potential of Cl¹⁻/Cl⁰,
the active composition contains substances for accelerating dissolving (effervescent salts), for example, a carbonate or bicarbonate-containing compound, such as, for example, sodium carbonate or sodium bicarbonate, and at least one stoichiometric amount of acid,
the agent contains a binder and optional flavoring, coloring and additive substances such as substances for softening water, fillers and the like,
the active composition is mixed or combined with the adhesive composition wherein the adhesive composition is suitable for adhering to the skin (i.e., external topical application) and/or to the mucous membranes,
the adhesive composition contains
at least paraffins, fats and/or oils
at least one water-soluble polymer, that may be selected from the group of cellulose derivatives and polysaccharides and
at least one alkyl vinyl ether/maleic anhydride copolymer,
the adhesive composition contains at least one alcohol,
the at least one alcohol is a liquid at room temperature and/or body temperature, ethanol being advantageous,
the at least one alcohol is added in an amount of up to 10% by weight, based on the total amount of the adhesive composition,
the at least one water-soluble polymer contains at least one polysaccharide, in particular the polysaccharide xanthan gum,
the at least one polysaccharide is added in an amount of up to 20% by weight, based on the total amount of the adhesive composition,
no fluoride salts are present, such as no fluoride compounds,
the agent is in the form of a cream, a paste or a gel which is suitable in particular for adhering to the skin (i.e., with external topical application),
the mucous membranes and/or the gingiva after application,
for treatment, a layer of at least 0.2 mm, such as at least 0.3 mm, or at least 0.5 mm, or at least 0.7 mm, or at least 1.0 mm, is applied,
the agent is spread smoothly with water after application to an area to be treated,
the active composition is in the form of a powdered solid mixture and is supplied as a powder for use and/or
the active composition in the form of a powdered solid mixture is mixed with the adhesive composition and supplied as a cream, paste or gel for use.

In addition, an agent, in particular a cosmetic and/or pharmaceutical agent, is disclosed, containing at least one organic or inorganic chlorine compound and at least one oxidizing agent, characterized in that it is present in the form of a cream and/or an ointment, a paste or a gel, which is suitable for adhering to the mucous membranes or the gingiva after application to the skin (e.g., with external topical application).

This also discloses an adhesive composition, which is suitable in particular as a vehicle for an agent with an active composition as described herein. The adhesive composition contains at least one paraffin, fat and/or oil, at least one water-soluble polymer, as may be selected from the group of cellulose derivatives and polysaccharides, at least one alkyl vinyl ether/maleic anhydride copolymer and at least one alcohol.

The adhesive composition is additionally advantageously characterized in that
the alcohol is present as a liquid at room temperature and/or at body temperature, ethanol being particularly desirable,
the at least one alcohol is added in an amount of up to 10% by weight, based on the total amount of the adhesive composition,
the at least one water-soluble polymer contains the polysaccharides xanthan gum,
the at least one polysaccharide is added in an amount of up to 20% by weight, based on the total amount of the adhesive composition.

In addition, a kit is disclosed, in particular a kit for use after a dental cleaning treatment, comprising at least one first package of a first dose of the agent according to the invention and at least one second package of a second dose of the agent according to the invention. At least one first package may comprises a combination of the active composition with the adhesive composition. The active composition or advantageously the combination of the active composition with the adhesive composition of the at least one first package is expediently supplied in the form of a paste, a cream, an ointment or a gel. This first package is used in particular immediately after a dental cleaning treatment. Because of the adhesive composition, after the agent according to the invention is applied, an effect that is prolonged over time and is therefore intensive is achieved, e.g., lasting 1 to 3 hours.

The at least one second package may be free of the aforementioned adhesive composition or contains a smaller amount of the aforementioned adhesive composition than the first package. The active composition of the at least one second package is expediently supplied in the form of a powder, a tablet, in particular in the form of a water-soluble powder and/or a water-soluble tablet, a paste, in particular a toothpaste or a solution, in particular an aqueous solution. Said solution may be relatively highly concentrated and can optionally be diluted further in water. The at least one second package is included several times in the kit, or at least three times, or at least five times. One of the second packages can be used on each of the days following the treatment—for example, once a day, by applying the respective dose by means of a toothbrush, for example.

Additional features, advantages and goals of the present invention are derived from the following description. The advantageous embodiment variants described below lead to further improvements in the agent according to the invention either alone or in combination with one another.

### BRIEF DESCRIPTION OF THE FIGURES

FIG. 1 shows a biofilm-inhibiting and destroying effect of an ointment according to the invention.

FIG. 2 shows microbial gingival infection over the treatment period.

FIG. 3 shows a summary of a plaque index progression over the treatment period.

FIG. 4 shows a summary of the gingival index progression over the treatment period.

FIG. 5 shows a summary of the periodontal bleeding index progression over the treatment period.

FIG. 6 shows a halitosis perception after 11 days.

### DETAILED DESCRIPTION OF THE INVENTION

The active composition is characterized in that it comprises at least one organic or inorganic chlorine compound and at least one oxidizing agent, which may be selected from the group of persulfate compounds. This active composition has the advantage that, after application of the agent to an injured and/or inflamed location, chlorine oxide or hypochlorite which appears to be responsible for the healing effect of the agent, is formed due to contact with moisture (i.e., water). The adhesive composition—inasmuch as it is present, then forms a barrier layer in which the active pharmaceutical substances are present. However, these substances do not manifest their effect until moisture reaches them.

Within the scope of the present description, the term “active composition” is understood to refer to components of the agent according to the invention, which exhibit cosmetic and pharmaceutical efficacy either alone or in combination. However, the term “adhesive composition” is understood to refer to components, which can serve as a vehicle (carrier) and application medium for the active composition, either alone or in combination. However, the adhesive composition also has the purpose of forming a barrier layer above the location to be treated (e.g., a fissure and/or injury and/or optionally an inflammation) and releasing the active ingredients only gradually. A localized application and adhesive of the agent according to the invention due to the adhesive composition permits a local and thus targeted use of the active ingredients.

The adhesive composition expediently has an adhesive strength of more than 7 N (Newtons), or of more than 7 N (in particular in measurements according to the ISO Standard 10873:2010, in particular in a one-minute test and/or in a 10-minute test at 25° C.).

The adhesive composition expediently has a viscosity in the range of 20 to 50 mPas (millipascal-seconds), or of 25 to 45 mPas (in particular measured with a Brookfield viscometer, expediently at 100 rpm with a spindle (cylinder) no. 4 and at 25° C., in particular with a mixture of 5 g propylene glycol and 1 g of the paste according to the invention).

The adhesive composition expediently has a consistency value in the range of 10 to 50 mm (millimeters), or of 20 to 40 mm (in particular in measurement according to BE EN 30193-1:1994, in particular at a weight of 1 kg at 25° C.).

The chlorine atom may be present in the starting compound, either in the form of an alkali salt or an alkaline earth metal salt or some other compound that releases chloride ions in an aqueous solution or may be bound to an organic radical. Organic molecules capable of releasing chloride ions include, for example, chloramine B (N-chlorobenzenesulfonamidosodium), chloramine T (p-toluenesulfonic chloramine sodium), dichloramine T (p-toluenesulfonic dichloramide), halazon (p-dichlorosulfamylbenzoic acid), dichlorocyanuric acid, trichlorocyanuric acid, TCM (trichloromelamine), 1,3-dichloro-5,5-dimethylhydantoin, dichloroglycoluril, succinic chlorimide or chloroazodin (N,N′-dichloro diazo dicarbonamidine). A mixture of the aforementioned chlorine-containing substances may also be used.

The agent according to the invention may be characterized in that it does not contain any fluoride salts, such as no fluoride compounds at all.

An oxidizing agent which capable of splitting off chlorine or chlorine oxide from the chlorine compound in an aqueous solution is advantageously used. Suitable oxidizing agents here include, for example, persulfate compounds, in particular hydrogen peroxosulfate compounds. Chlorine (Cl₂) or dichlorine oxide (Cl₂O⁻) may be formed in combination with at least one suitable oxidizing agent by reaction with dissolved chloride ions (Cl⁻). The advantage of such an active composition is that the chlorine is released gradually. The oxidation potential of the oxidizing agent in solution is advantageously higher than the oxidation potential of Cl⁻/Cl⁰ in solution at least in the desired pH range of <6 and/or <5, so that the desired reaction can occur.

Although fundamentally different oxidizing agents may be used, the active composition used as the oxidizing agent advantageously contains a persulfate compound, in particular a hydrogen peroxosulfate compound. Persulfate compounds include peroxomonosulfate compounds (i.e., compounds containing the (SO₅)²⁻ anion) and peroxodisulfate compounds (i.e., compounds containing the (S₂O₈)²⁻ anion). Examples of these compounds include peroxomonosulfates, such as Na₂SO₅ or KHSO₅ and peroxodisulfates such as Na₂S₂O₈. The hydrogen peroxosulfate compounds are especially advantageous as the oxidizing agent. One advantageous agent is obtained with potassium hydrogen monopersulfate (KHSO₅) as the oxidizing agent and a salt form of the chloride compound, such as from the group of alkali salts, for example, table salt (NaCl). This has proven to be especially effective.

The agent according to the invention, in particular the active composition, expediently contains a carboxylic acid, such as a monocarboxylic or dicarboxylic acid such as citric acid. These organic carboxylic acids ensure an acidic pH, which can further improve the efficacy of the agent.

An advantageous active composition, which can be prepared inexpensively, contains potassium hydrogen monopersulfate (KHSO₅), table salt (NaCl) and an acid, such as in the form of a monocarboxylic acid or a dicarboxylic acid, such as oxalic acid, tartaric acid, succinic acid or citric acid. The active composition may contain an amount of acid, such that, after the active composition has dissolved in an aqueous medium (5 g active composition in 50 mL water), its pH will be <5, or <4.5. Citric acid is completely safe physiologically, degrades rapidly in nature and therefore may be for use in the active composition.

The active composition may additionally contain antimicrobial compounds such as potassium monopersulfate, potassium carbonates, sodium peroxycarbonate, sodium benzoate, subtilisin, potassium benzoate, chlorhexidine, a combination of chlorhexidine and thymol, cetyl pyridinium chloride, halogen-cleaving compounds such as PVP iodine and cyanuric acid chloride and/or formaldehyde-cleaving compounds such as paraformaldehyde and/or methylol compounds, etc. Furthermore, analgesic active ingredients such as lidocaine, polidocanol or benzydamine are also advantageously used.

The active composition expediently contains a binder and optional flavoring agents, coloring agents and additives such as substances to soften hard water, fillers and the like. Examples of binders that may be used include modified cornstarch, microcrystalline cellulose, sorbitol, hydrogenated soy triglycerides, polyethylene glycol, such as PEG 180, PEG 150, PEG 75, polyvinylpyrrolidones, a copolymer of ethylene oxide and propylene oxide, polyvinylpyrrolidone or a copolymer of polyvinylpyrrolidone and vinyl acetate. The amount by weight of the binder is expediently max. approx. 30% by weight and may be between 5% and 30% by weight, or between 5% and 25% by weight of the total weight of the active composition. If an adhesive composition can be used in combination with the active composition then it is optionally possible to omit any other binder as described above because the adhesive composition described herein completely absorbs the active composition and/or at least one of its ingredients assumes the binding function for the active composition.

The adhesive composition may contain at least one water-soluble polymer, as may be selected from the group of cellulose derivatives and at least one alkyl vinyl ether/maleic anhydride copolymer and optionally refined paraffins (liquid petrolatum), fats and/or oils. The adhesive composition may additionally optionally contain zinc-containing substances in particular zinc oxide, silicon dioxide and polyethylene glycol. Possible compositions of this type and similar compositions which can be used as adhesive compositions are mentioned in the following patents and patent applications: U.S. patent application Ser. No. 13/574,230, U.S. patent application Ser. No. 13/574,224, U.S. Pat. Nos. 4,758,630 and 5,561,177, EP patents EP 2 525 768 and EP 2 525 769. The contents of the aforementioned documents are herewith included by reference to the present patent specification.

The active composition may be added to the adhesive composition in the form of a powdered solid mixture. This form of addition may have a retarding effect on the development of the active ingredient, for example, by the fact that the powdered active composition dissolves only by coming gradually in contact with saliva, for example.

The active composition may optionally contain a surfactant. Fundamentally various surfactants known to those skilled in the art may be used. If carboxylic acids are used, then the surfactants should be stable at a pH of less than 7. The term “stable” in this context should be understood to mean that max. 10% of the surfactant that is used will disintegrate in solution at room temperature within 30 minutes. Fatty alcohol-polyglycol ethers, alkylbenzene sulfonates, alkyl sulfonates may be used as the surfactants. Anionic surfactants, such as from the group of alkyl ether sulfates such as fatty alcohol ether sulfate alkali salts, for example, sodium n-alkyl-C₁₂₋₁₄-diglycol ether sulfate or sodium n-alkyl-C₁₂₋₁₄-diglycol ether sulfate or sodium n-alkyl-C₁₂₋₁₄-diglycol ether sulfate have proven especially suitable. Also suitable, for example, Na lauryl sulfate, Na lauryl sulfoacetate, trisodium phosphate.

An example of an active composition contains the following components:

| | | Desired |
|---|---|---|
| | In wt % | ranges |
| Substance | (%) | In wt % (%) |
| Citric acid | 30 | 15 to 50 |
| Lauryl sulfate (in particular sodium lauryl sulfate) | 15 | 2 to 30 |
| Sodium bicarbonate and/or sodium carbonate | 20 | 10 to 40 |
| Sodium chloride | 10 | 4 to 20 |
| Potassium hydrogen monopersulfate | 5 | 1 to 10 |
| Binder | 20 | 0 to 30 |

To prepare the active cosmetic and/or pharmaceutical agent (substance mixture), the active composition is expediently dispersed in an adhesive composition (i.e., an adhesive cream or an adhesive gel) and distributed finely until obtaining a homogeneous mixture. The adhesive composition and the active composition may be mixed together in a weight ratio between 99:1 and 80:20, or between 98:2 and 88:12.

The adhesive compositions which serve as the vehicle material for the active composition (i.e., for the active cosmetic and/or pharmaceutical components and/or composition) absorb the active composition and/or the ingredients thereof, for example, by mixing the adhesive compositions with the active composition to form a substance mixture, so that the adhesion properties of the adhesion composition are combined with the cosmetic and pharmaceutical effects of the active composition.

A particularly advantageous adhesive composition is presented below. The particularly advantageous adhesive composition is based on at least one oil or fat or a mixture thereof, such as of a vegetable type and at least one alkyl vinyl ether/maleic anhydride copolymer. The alkyl vinyl ether/maleic anhydride copolymer is advantageously in the form of an acid, ester and/or salt. The cations of the salts are usually selected from the group consisting of calcium, potassium, sodium, magnesium, aluminum, zinc salts and mixtures thereof, in particular from the group consisting of Ca²⁺, K⁺, Na⁺, Mg²⁺, Al³⁺ and/or Zn²⁺. In particular, a methyl vinyl ether/maleic anhydride copolymer is used as the alkyl vinyl ether/maleic anhydride copolymer. The methyl vinyl ether/maleic anhydride copolymer is present in an amount of 20-45% by weight, based on the total amount of the adhesive compositions, for example, as a salt, an ester and/or an acid. The methyl vinyl ether/maleic anhydride copolymer is advantageously in the form of a salt and/or acid, for example, in an amount of 25-45% by weight or 25-40% by weight or 28-40% by weight, based on the total amount of the adhesive compositions.

In addition, the adhesive composition contains at least one water-soluble polymer in particular selected from the group consisting of cellulose derivatives. The cellulose derivatives are water-soluble polymers, as may be selected from the group consisting of methyl cellulose, carboxymethyl cellulose, sodium carboxymethyl cellulose, hydroxypropylmethyl cellulose and mixture thereof. Carboxymethyl cellulose, in particular sodium carboxymethyl cellulose may be for use here. The water-soluble polymer selected from the group of cellulose derivatives is used in an amount of up to 45% by weight, based on the total amount of the adhesive compositions, for example. The water-soluble polymer may be selected from the group of cellulose derivatives in an amount of 2 to 40% by weight, based on the total amount of the adhesive compositions.

The adhesive composition additionally contains at least one water-soluble or swellable polymer selected from the group of polysaccharides, in particular xanthan gum, which serves as a thickener. Polysaccharides, which have an intrinsic viscosity, i.e., have a shear thinning effect are desired. Xanthan gum in particular swells in aqueous solution, thereby increasing the viscosity of the medium, and having a shear thinning effect. Polysaccharides, in particular xanthan gum, may be present in an amount of 0 to 20% by weight, or from 2 to 20% by weight, based on the total amount of the adhesive compositions.

The sum of the amount of cellulose derivatives and polysaccharides is advantageously in the range of 2 to 40% by weight, based on the total amount of the adhesive compositions. A weight ratio of polysaccharides, for example, xanthan gum to cellulose derivatives, of at least 1:1, or at least 2:1, has proven to be advantageous. The extent to which the cellulose derivatives are compared to the polysaccharides within the scope of the present patent application, the cellulose derivatives are not included in the group of polysaccharides.

The adhesive composition optionally also contains a liquid alcohol, in particular ethanol. The alcohol may be present in an amount of 0% to 10% by weight or from 2% to 8% by weight or from 3% to 7% by weight, based on the total amount of the adhesive composition. By adding the aforementioned alcohol, an improved binding of the adhesive composition is achieved.

The adhesive composition may contain at least one additional substance, selected from the group consisting of trihydroxystearin, silicon dioxide and phosphodiglycerides.

The adhesive composition may contain trihydroxystearin in an amount of 0.001-5.0% by weight and more preferably 0.1-4.5% by weight and even more preferably from 0.5 to 4.0% by weight, each based on the total amount of the adhesive composition. By adding trihydroxystearin, the adhesive properties of the adhesive composition and thus of the total agent according to the invention (substance mixture) are improved.

The adhesive composition optionally contains silicon dioxide in an amount of 0.001-5% by weight, or of 0.1-4% by weight, based on the total amount of the adhesive composition. The flowability and the consistency of the adhesive composition can be influenced in a positive sense by adding silicon dioxide. A tendency to liquefaction can be effectively prevented by silicon dioxide.

The adhesive composition optionally contains phosphoglycerides, preferably in an amount of 0.001-4% by weight, or of 0.01-3% by weight and even more preferably of 0.1-2% by weight, based on the total amount of the adhesive composition. The phosphoglycerides are selected in particular from the group of lecithins, especially soy lecithin, for example.

A desired adhesive composition is characterized in that at least one lecithin and trihydroxystearin are present in combination therein, optionally lecithin, trihydroxystearin and silicon dioxide being present in combination. Lecithin is advantageously present in an amount of 0.001-4% by weight, or 0.001-3% by weight, or of 0.01-2% by weight, and trihydroxystearin is present in an amount of 0.001-5% by weight, or of 0.1-4.5% by weight or of 0.5-4% by weight, each based on the total amount of the adhesive composition. If vegetable oils and/or fat is/are used, in particular with a certain minimal amount of unsaturated fatty acid, for the adhesive composition, then advantageous adhesive properties are achieved in particular when the adhesive composition contains substances selected from the group consisting of phosphoglycerides and trihydroxystearin.

Alternatively, silicon dioxide is present, optionally in combination with trihydroxystearin and/or a phosphoglyceride, in particular lecithin. In addition to the silicon dioxide, trihydroxystearin and polyethylene glycol may also lead to stabilized adhesive compositions. These substances yield good results for compositions based on vegetable oils and/or fats as well as those based on mineral oils and/or fats.

It is desireable for the total amount of the trihydroxystearin, silicon dioxide and phosphoglycerides present not to exceed the maximum amount of 15% by weight, or 12% by weight, or 10% by weight, each based on the total amount of the adhesive composition.

A particularly advantageous adhesive composition is characterized in that a vegetable and/or fat is present in an amount of at least 25% by weight, preferably at least 30% by weight, based on the total amount of the adhesive composition, wherein the fatty acid content of the vegetable oil and/or fat may consist of at least 20% by weight, at least 30% by weight, at least 65% by weight, or at least 80% by weight higher fatty acids with a chain length of 16-18 carbons.

A particularly advantageous adhesive composition is additionally characterized in that vegetable oils and/or fats are used whose fatty acid content consists of in particular at least 20% by weight, 40% by weight, 50% by weight, 60% by weight, 70% by weight, or 80% by weight unsaturated fatty acids. This offers the advantage that the adhesive composition is particularly tolerable when swallowed, which may be advantageous in particular for use on children.

The amount of vegetable oil and/or fat has an influence on the consistency of the adhesive compositions. If too little is added, then the adhesive composition and thus the entire substance mixture (containing the active composition and the adhesive composition) may assume a granular dry consistency. Adding silicon dioxide, trihydroxystearin and phosphoglycerides can counteract this.

One embodiment of adhesive composition contains, for example, based on the total amount of the adhesive composition:

a) 25-60% by weight of at least one oil and/or fat, such as of a vegetable type,

b) 2-40% by weight of at least one water-soluble polymer selected from the group of cellulose derivatives and polysaccharides,

c) 25-45% by weight of the at least one alkyl vinyl ether/maleic anhydride copolymer,

d) 0-10% by weight or optionally up to 10% by weight of an alcohol,

e) 0-5% by weight or optionally up to 5% by weight trihydroxystearin,

f) 0-4% by weight or optionally up to 4% by weight phosphoglycerides,

g) 0-5% by weight or optionally up to 5% by weight silicon dioxide and

h) 0-10% by weight of additional additives.

The additional additives include, for example, in particular, flavorings (such as, e.g., menthol, citrus lemon, peppermint flavoring and menthyl lactate), lactose monohydrate, zinc oxide (ZnO), petrolatum, paraffins and liquid petrolatum. Other possible additives include, for example, antioxidants, coloring agents, stabilizers, thickeners, emulsifiers, etc. A substance may belong to several active groups here and/or manifest several effects.

Zinc oxide can influence the adhesive power and adhesive time, in particular increasing it. Zinc oxide should be omitted for applications in the area of the mouth and throat. It is generally desireable to limit the zinc content to an upper limit of max. 1% by weight, based on the total amount of the adhesive composition, and/or it is desireable not to use any zinc additives.

The addition of paraffins and liquid petrolatum is optional. The paraffin and liquid petrolatum content may be limited to max. 3% by weight total, or max. 1% by weight, based on the total amount of the adhesive composition. Paraffin or liquid petrolatum may be excluded in particular for applications in the area of the mouth and throat.

The addition of petrolatum is optional. The petrolatum content may be limited to max. 2% by weight, or to max. 0.5% by weight, based on the total amount of the adhesive composition. Advantageously no petrolatum is added, in particular for applications in the area of the mouth and throat.

The addition of lactose monohydrate is optional. It may serve as a vehicle for flavoring or for the active ingredient. It promotes the consistency of the mixture and leads in particular to a creamy consistency. The lactose monohydrate content may be limited to a total of max. 1% by weight, or to max. 0.5% by weight, based on the total amount of the adhesive composition.

A portion of the viscous agent is applied to the area to be treated (in particular a wound and optionally an inflamed area) and distributed over the area by the user/patient, so that the border zone of the area to be treated is also covered well. The agent forms a barrier layer or protective layer on the area to be treated. At the same time, the active pharmaceutical ingredients present in the protective layer manifest their effect. The powdered components present in the agent are dissolved in the moisture originating from the skin, the mucous membranes and/or the wound and moisture optionally present in the environment, so that these can react with one another. A good long-term effect can be achieved because these components are only slowly dissolved.

An agent for topical treatment advantageously includes a flowable pasty adhesive composition, which is suitable for adhering to the area to be treated after application and also contains an active pharmaceutical composition with an organic or inorganic chlorine compound and at least one oxidizing agent. If the organic or inorganic chlorine compound and the oxidizing agent go into solution, chlorine or chlorine oxide is formed, which has been found to be particularly effective in the treatment of wounds and optionally inflammations of a wide variety of types. The active composition advantageously contains an organic carboxylic acid to adjust a pH of <6.

The invention will now be explained in greater detail on the basis of examples.

### APPLICATION EXAMPLES

### Application Example A

The following table shows an agent according to the invention in the form of a paste, an ointment or a cream for topical application to chapped or cracked skin areas, in particular for use on the soles of the feet, heels, toes, hands, palm, fingers, corner of the mouth, sides of the nose.

| | | Percent by |
|---|---|---|
| Ingredients | CAS No. | weight (wt %) |
| Ca/Na PVM/MA copolymer | 62386-95-2 | 37.978 |
| Olea Europa (olive oil) | 8001-25-0 | 30.058 |
| Xanthan gum | 11138-66-2 | 11.116 |
| Alcohol | 64-17-5 | 5.187 |
| Carboxymethyl cellulose (CMC) | 9004-32-4 | 3.705 |
| Trihydroxystearin | 139-44-6 | 3.578 |
| Silicon dioxide | 68611-44-9, | 3.110 |
| | 2945-52-5 | |
| Lecithin | 8002-43-5 | 1.017 |
| Zinc oxide | 1314-13-2 | 0.926 |
| Liquid paraffin | 8042-47-5 | 0.642 |
| Citric acid | 77-92-9 | 0.599 |
| Sodium lauryl sulfate | 151-21-3 | 0.400 |
| Lactose monohydrate | 10039-26-6 | 0.400 |
| Menthol | 2216-51-5 | 0.276 |
| Petrolatum | 8009-03-08 | 0.271 |
| Sodium bicarbonate | 144-55-8 | 0.240 |
| Sodium chloride | 7647-14-5 | 0.200 |
| Potassium hydrogen monopersulfate | 70693-62-8 | 0.100 |
| Citrus lemon | 8008-56-8 | 0.066 |
| Sodium carbonate | 144-55-8 | 0.060 |
| Peppermint flavoring | — | 0.040 |
| Menthyl lactate | 59259-38-0 | 0.031 |

Flavoring substances, in particular peppermint flavoring, menthyl lactate, citrus lemon, menthol and mixtures thereof, are optional and/or may be replaced.

The paste may be characterized by the following properties:

1. Adhesive properties tested according to the ISO standard 10873:2010 Dentistry—Denture adhesive section 7.3 Determination of stability—Aging procedure) (conditions: test (peel and adhesion test) (a) 1 min, 25° C., (b) 10 min, 25° C.).

Adhesion strength: (a)>7.6 N

(b)>7.6 N

2. Measurement of viscosity with a Brookfield viscometer (conditions: 5 g propylene glycol, 1 g of the paste according to the invention; 100 rpm with spindle no. 4 at 25° C.). Viscosity: 35±5 mPas.

3. Measurement of consistency according to BE EN 30193-1:1994 (Dentistry standard for resilient lining materials for removable dentures, conditions: weight 1 kg, 25° C. Value for the consistency: 20-40 mm (i.e., the average propagation diameter (in millimeters) of an amount of 1.5 g of the cream to be tested between two glass plates after 5 minutes, wherein the top plate is loaded with a weight of 1 kg).

### Application Composition B

The following table shows the agent according to the invention in the form of a powder for topical application:

| | | Percent by weight |
|---|---|---|
| Ingredients | CAS No. | (wt %) |
| Citric acid | 77-92-9 | 30 |
| Sodium lauryl sulfate | 151-21-3 | 15 |
| Sodium bicarbonate | 144-55-8 | 20 |
| Sodium chloride | 7647-14-5 | 10 |
| Potassium hydrogen monopersulfate | 70693-62-8 | 5 |
| Binder | | 20 |

### Example 1

Finding: Cracked skin approx. 2 mm wide, 10 mm long and 2 to 3 mm deep, on the right heel.

Treatment: Three applications of the compositions according to the application composition A on five consecutive days, applied to the heel.

Method: The applied cream was covered with water after application.

Healing process: Immediate pain relief. After 5 days, the cracked skin had healed completely.

### Example 2

Finding: Rough dry cracked skin, hardened for 20 years on the heels with multiple fissures with a depth of approx. 2 mm.

Treatment: Three applications each of the composition according to the application composition A on four consecutive days (i.e., a total of 12 applications) to the heels.

Method: The cream was covered with a dressing after application.

Healing process: On the fourth day, the hardening had disappeared and the fissures were closed and largely healed.

### Example 3

General condition: A diabetic patient who had already lost all his fingers.

Finding: Diabetic patient with a surgical wound (diameter approx. 2-3 mm) on his left foot after amputation of one toe. Regular cleaning of the wound with a disinfectant and subsequent dressing of the wound. No healing had occurred after more than 4 weeks post-operatively. The wound is infected, leaking pus.

Treatment: A single application of the application composition B (in the form of a powder).

Method: Cleaning the wound with a disinfectant and dusting the wound with the powder (approx. 2-3 g of the application composition B). Covering the wound for 2 days with a dressing. Regular cleaning with a disinfectant, starting on the second day after the single application of the application composition B.

Healing process: Immediately after application the patient did not feel any irritation. On the second day after application, a dark fluid was observed running out of the application site. At the same time, the patient felt an irritation. The wound had successively closed on the following days.

### Example 4

Finding: Painful skin ulcer on the left thumb.

Treatment: Application of a cream according to the application composition A on two consecutive days.

Method: The cream was covered with an adhesive dressing after being applied.

Healing process: The cream disappeared after the first night. After 2 days, the skin ulcer had also disappeared.

### Example 5

Finding: Painful skin ulcer on the right thumb.

Treatment: Application of a cream according to the instructions for use A on four consecutive days.

Method: The cream was covered with an adhesive dressing after being applied.

Healing process: The pain had subsided after 3 days. The skin ulcer was definitely smaller after 1 week.

### Example 6

Finding: Fine skin fissure for a length of approx. 5-7 mm in the fold of the outer ear.

Treatment: A single application of the cream according to the application composition A.

Method: Application of the cream according to the application composition A and spreading of the applied cream with a wet finger.

Healing process: Immediate pain relief. After about 12 hours, the cream has dried out at the surface and covers the area with the cracked skin. After about 24 hours, the remaining cream was washed off. The cracked skin could no longer be discerned, i.e., it has largely healed.

### Example 7

Finding: Patient with or without chronic periodontitis.

Treatment: One application of the application composition A after a regular dental cleaning treatment, in particular after a tooth and pocket cleaning, in particular after a scaling and root planing (SRP) treatment.

Method: Application of the cream according to the application composition A to the gingiva, in particular to the gingival margin.

Results: The application composition A relieves pain and promotes the healing process after the treatment (in particular of small injuries that occur during the treatment), providing protection for a period of time after the treatment by covering the treated areas and reducing or preventing gingival bleeding after the treatment.

### Example 8

Study for evaluating the biofilm-inhibiting and biofilm-destroying activity on the example of the application composition A.

The goal of the study was to evaluate the biofilm-inhibiting and biofilm-destroying activity of an ointment against Staphylococcus aureus. To achieve this goal, an in vitro model system, which simulates the conditions found in wounds in vivo, was used.

a) Materials and Methods:

1. Strains and Culture Conditions

Staphylococcus aureus Mu50 was cultured aerobically on Luria Bertani (LB) agar (BD, Sparks, Md.) at 37° C. S. aureus Mu50 is a methicillin-resistant strain of S. aureus.

2. Tested Product

The ointment A (i.e., application composition A) was evaluated by distributing approx. 250 mg aliquots on the artificial dermis.

3. Producing the Artificial Dermis

The artificial dermis comprises a top layer and a bottom layer. The top layer is a chemically crosslinked spongy hyaluronic acid (HA) layer and was produced as described above with minor modifications (Mineo et al., 2012). Sodium hyaluronate powder (1.20-1.80 MDA, Lifecore Biomedical, MN, US) was dissolved in distilled water (DW) at a concentration of 1.5% (pH 6.8). This high-molecular HA (HMWA) solution was adjusted to a pH of 3.5 with a dilute HCl solution. Ethylene glycol diglycidyl ether (EX810; Sigma Aldrich, Bornem, Belgium) was used as the crosslinking agent for HA molecules. An aqueous EX810 solution was added by drops to the HMW-HA solution while stirring vigorously (weight ratio of HA to EX810: 5-1). This mixed solution was poured into a freeze-drying container, stored at 4° C. overnight, frozen at −80° C. and then freeze dried. This sponge was carefully rinsed with DW to remove any free crosslinking agent, then frozen at −80° C. and freeze dried to obtain the spongy crosslinking HMW-HA layer (cHMW-HA). The bottom layer is a spongy layer consisting of HA and collagen (Col). HA powder was dissolved in DW at 1% concentration in order to prepare HMW-HA solution (pH 6.8). Separately HA (1%) was autoclaved at 120° C. for 1 hour to obtain a partially hydrolyzed low-molecular HA (LMW-HA) solution. A 0.1% Col-containing collagen solution was prepared, heated for 10 minutes at 50° C. to obtain a heat-denatured collagen solution. The three solutions mentioned above were mixed and then adjusted to a pH of 7.5 using NaOH. This mixed solution was poured into a freeze-drying container in which the spongy cHMW-HA layer was immersed. The combined product was stored overnight at 4° C., then frozen at −80° C. and freeze dried to obtain a two-layer spongy layer. Both sides of the spongy layer were irradiated with a UV lamp for 20 minutes to crosslink the collagen molecules. The spongy layer was sterilized for 1 hour at 100° C. to obtain a sterile artificial dermis (AD).

4. Chronic In Vitro Wound Model for Evaluating the Inhibiting Effect of the Ointment and the Biofilm-Destroying Effect

Biofilms were cultured in a chronic pathogenic wound biofilm model. An overnight culture of the tested strain was pelletized, washed, resuspended in physiological saline solution (PS) and diluted to 106 CFU mL⁻¹ (colony-forming units per milliliter). The AD was steeped in 0.5 mL chronic wound biofilm medium (Bolton broth with 50% plasma and 5% hemolyzed frost-thaw horse blood and 10 U/mL heparin) and placed in the cavities (English: wells) in a 24-well microtiter plate (TPP) and 10 μL aliquots of the cell suspension were applied to each AD. Then 0.5 mL chronic wound biofilm medium was added to each cavity (i.e., each well). To evaluate the biofilm inhibiting effect of the ointment, 250 mg aliquots were applied to the AD immediately after inoculation and the biofilm model was incubated for 24 hours at 37° C. in order to allow a biofilm to form on the AD.

To evaluate the biofilm-destroying activity of the ointment, the biofilm model is incubated for 24 hours at 37° C. to allow a biofilm to form on the AD. After this formation of biofilm, the medium was removed and the biofilm was washed with PS, fresh medium was added and ointment was placed on the biofilm.

5. Plating Out and Data Analysis

In both types of experiment, after being treated, both types of films were rinsed with PS in order to remove the treatment. The AD was placed in 10 mL PS. Sessile cells were removed from the AD with three cycles of agitating (30 s) and ultrasonic bombardment (30 s; Branson 3510; Branson Ultrasonics Corp., Danbury, Conn.) and the number of CFU/AD (/mL) was determined by plating out the resulting suspensions. The normal distribution of the data was tested using the Shapiro-Wilk test. The data was then analyzed by an independent statistical t-test sample analysis. Statistical analysis and linear regression analysis were performed using SPS software version 22.0 (SPSS, Chicago, Ill., USA).

b) Result:

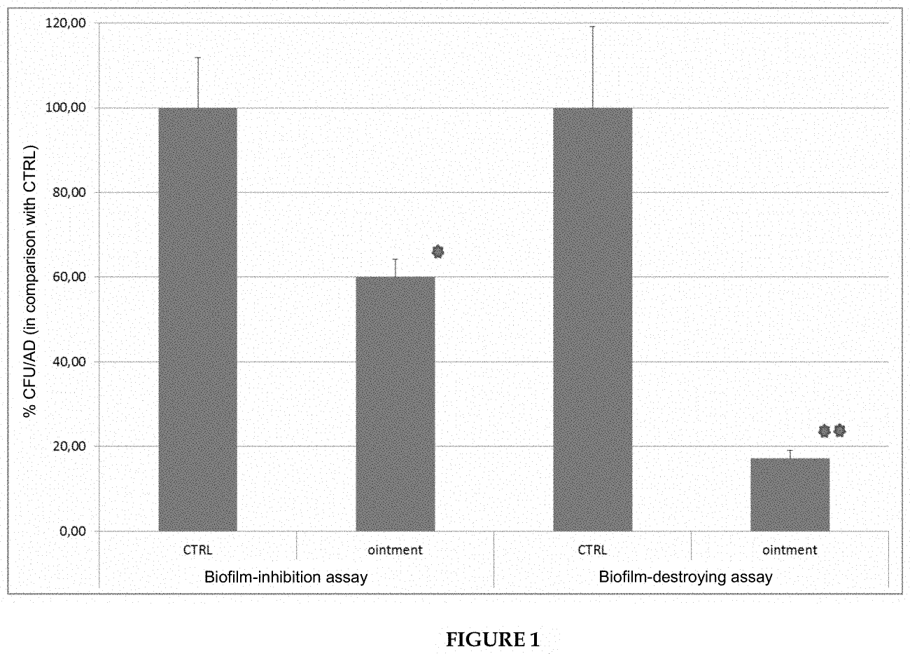

The biofilm-inhibiting and biofilm-destroying effects of the ointment against S. aureus Mu50 can be seen in FIG. 1, Table 1 and Table 2. The ointment had a low but significant (p<0.01) biofilm-inhibiting effect. The treatment with the ointment during formation of the biofilm led to a 40% reduction in the number of CFU/AD. In addition, the ointment had a moderate biofilm-destroying activity. Treatment of a mature biofilm with the ointment led to an 82.8% reduction in the number of CFU/AD.

TABLE 1
Inhibitory effect of the ointment on S. aureus Mu50

| | CFU/AD | | Log CFU/AD | | % CFU/AD | |
|---|---|---|---|---|---|---|
| Treatment | ø | SD | ø | SD | ø | SD |
| CTRL | 1.87E+08 | 2.20E+07 | 8.27 | 0.05 | 100.0 | 11.8 |
| (without | | | | | | |
| treatment) | | | | | | |
| Ointment | 1.12E+08* | 8.00E+06 | 8.05* | 0.03 | 60.0* | 4.29 |

*Differs significantly from the untreated control (p < 0.01)

The data in the table is given as the mean (CFU/AD)±standard deviation, mean log values (CFU/AD)±standard deviation and mean % value CFU/AD (in comparison with CTRL).

TABLE 2
Biofilm-destroying effect of the ointment on S. aureus Mu50

| | CFU/AD | | Log CFU/AD | | % CFU/AD | |
|---|---|---|---|---|---|---|
| Treatment | ø | SD | ø | SD | ø | SD |
| CTRL | 2.13E+08 | 4.11E+07 | 8.33 | 0.08 | 100.0 | 19.2 |
| (without | | | | | | |
| treatment) | | | | | | |
| Ointment | 3.67E+07* | 4.04E+06 | 7.56* | 0.05 | 17.2* | 1.9 |

*Differs significantly from the untreated control (p < 0.01)

The data in the table is given as the mean (CFU/AD)±standard deviation, mean log values (CFU/AD)±standard deviation and mean % value CFU/AD (in comparison with CTRL).

These results are also illustrated in FIG. 1. FIG. 1 presents the biofilm-inhibiting and destroying effect of the ointment. The data is given as the mean percent value CFU/AD (in comparison with the untreated CTRL)±standard deviation. In the biofilm inhibition assay, the product led to a significant (p<0.05) decline in the biofilm formation in comparison with the untreated control (labeled as CTRL) (* in FIG. 1). In the biofilm-destroying assay, the product led to a significant (p>0.005) decline in a mature biofilm in comparison with the untreated controls (labeled as CTRL) (** in FIG. 1).

### Example 9

Multicenter Open Randomized Clinical Trial

A multicenter open randomized clinical trial was carried to evaluate and compare the efficacy and safety of the agent according to the invention in the form of a gingival paste dressing according to the application composition A during a test phase I and in the form of a gingival brush solution prepared from the application composition B during a test phase II, wherein the two forms of the agent according to the invention were used on volunteers suffering from chronic periodontitis, as an additional application after a prior scaling and root planing (SRP).

9.1 Description of Volunteers

A total of 29 male and female volunteers and/or patients (ages 30 to 50), who were suffering from periodontitis and required an SRP treatment, were examined and registered for the study. Of these volunteers, 20 volunteers were randomly included in a treatment group and 19 volunteers were randomly included in a control group.

9.2 Treatment

Treatment of the Treatment Group:

Treatment with scaling and root planing (SRP) followed by phase I and phase II:

Phase I: Immediately after the SRP treatment of volunteers suffering from chronic periodontitis, the gingival paste (application composition A) was applied over the gingiva once and applied to the gingival sulcus for 2 to 3 hours.

Phase II: Phase I was followed by brushing the teeth and gingiva with the gingival brush solution once a day each evening after the last meal of the day, tested on 10 successive days beginning with the day after the SRP treatment. The gingival brushing solution was prepared fresh each day from the application composition B: one tablet (approx. 2 g) of the application composition B was dissolved in approx. 15 mL water immediately before use.

The duration of the treatment including phase I and phase II was 11 days.

Treatment of the Control Group:

Only treated with scaling and root planing (SRP).

9.3 Testing Schedule

Both treatment groups were evaluated as follows on day 0, day 5 and day 11:
before SRP treatment on day 0 (or up to 5 days earlier): demographic data, physical examination, vital parameters, performance parameters,
on day 0 after SRP treatment (control group) or after phase I treatment (treatment group): physical examination, vital parameters, performance parameters,
on day 5: physical examination, vital parameters, clinical periodontal parameters and performance parameters,
on day 11: physical examination, vital parameters, clinical periodontal parameters and performance parameters, total bacterial count and analysis, efficacy was evaluated by determining subgingival plaque.

Parameters measured at each evaluation (performance parameters):
plaque index (PI)
gingival index (GI)
periodontal bleeding index (PBI)

Bacterial count of periodontal phytogenic bacteria:
Prophyrompnas gingialis (Pg)
Prevotella intermedoa (Pi)
Aggregatibacer actinomycetemcomitans (Aa)
Fusobacterium nucleatum (Fn)

9.4 Test results

9.4.1 Microbial Gingival Infection Over the Treatment Period

Control group: 66.7% were infected at the start of the treatment. Of these 66.7, 100% were still infected even after 11 days.

Treatment group: 60% were infected at the start of the treatment. Of these 60%, only 25% were still infected after 11 days.

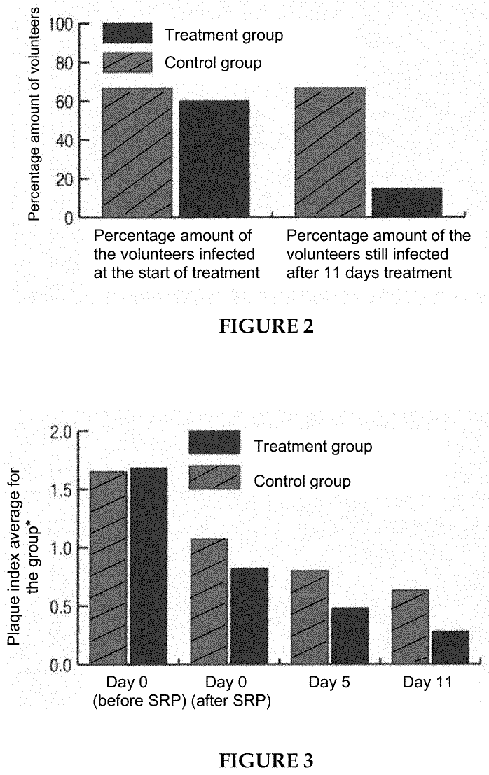

In a comparison of the control group and the treatment group, FIG. 2 shows on the left side of the diagram the percentage amount of volunteers in each group who were infected at the start of the treatment and on the right side of the diagram the percentage amount of volunteers of each group who were still infected on day 11.

After complete treatment on day 11, a large number of volunteers in the treatment group were free of infection whereas the control group still had the same number of infected volunteers as at the start of the treatment.

9.4.2 Summary of the Plaque Index Progression Over the Treatment Period

In a comparison of the control group and the treatment group, FIG. 3 shows the average plaque index for each group on day 0 before SRP, on day 0 after SRP and phase I treatment, on day and finally on day 11.

The plaque levels were evaluated with the Turkesy modification of the Quiglet-Hein plaque index (* in FIG. 3).

During the treatment period of 11 days, the plaque level declined in both groups but to a much lower level in the treatment group than in the control group.

9.4.3 Summary of the Gingival Index Progression Over the Treatment Period

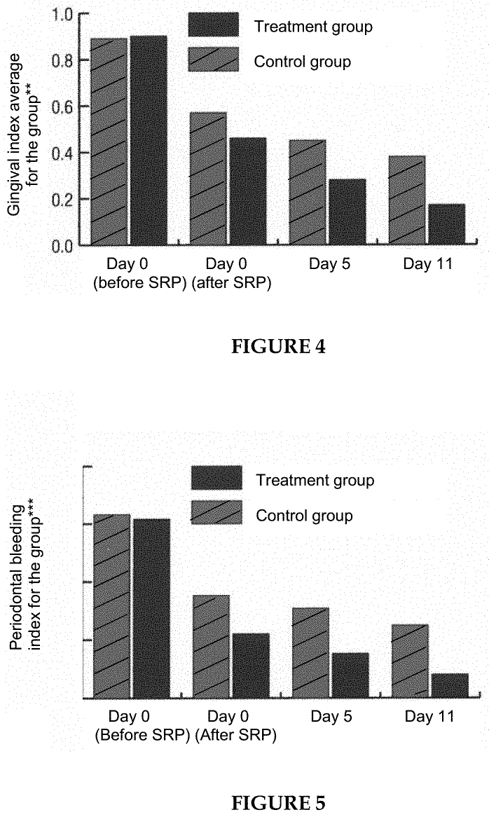

In a comparison of the control group and the treatment group, FIG. 4 shows the average gingival index for each group on day 0 before SRP, on day after SRP and after phase I treatment, on day 5 and day 11.

The gingival index was evaluated with the Loe and Silness 1963 index system for evaluation of the gingival condition, while qualitative changes in the gingiva were recorded (** in FIG. 4). The marginal and interproximal tissue was evaluated separately on the basis of 0 to 3.

During the treatment period of 11 days, the gingival level declined in both groups but was reduced to a much lower level in the treatment group than in the control group.

9.4.4 Summary of the Periodontal Bleeding Index Progression Over the Treatment Period

In a comparison of the control group and the treatment group, FIG. 5 shows the periodontal bleeding index average for each group on day 0 before SRP, on day after SRP and phase I treatment, on day 5 and on day 11.

The periodontal bleeding index was measured using the Muhleman and Son 1971 score chart which describes the bleeding of the sulcus bleeding (*** in FIG. 5).

During the treatment period of 11 days, the periodontal bleeding level declined in both groups but to a much lower level in the treatment group than in the control group.

9.4.5 Halitosis Feeling after 11 Days

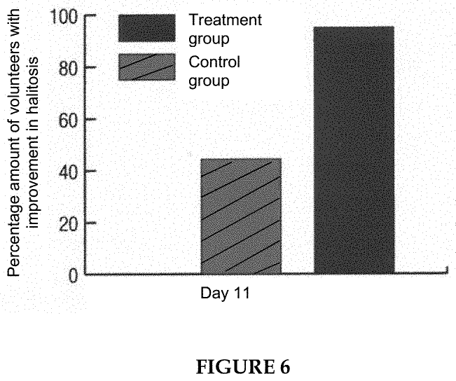

In a comparison of the control group and the treatment group, FIG. 6 shows the percentage of volunteers who experienced a decline in the halitosis perception for each group on day 11.

100% of the volunteers in the treatment group experienced a decline in the perception of halitosis whereas in the control group only approx. 40% of the volunteers experienced a decline.

9.5 Safety Results

No serious negative results were reported during the study period. None of the volunteers was excluded from the study because of any negative results. The overall test treatment was tolerated well by all the study volunteers.

9.6 Conclusion

None of the volunteers in the treatment group complained of any irritation after applying the periodontal paste dressing. Internal comparisons showed that the volunteers in the treatment group experiences statistically significantly greater declines in the plaque index, the gingival index and the periodontal bleeding index after 11 days.

The application of the periodontal paste dressing reduced the bacterial load on the gingiva substantially in comparison with the control group on day 11. A total of 9 of 11 volunteers (82%) in the treatment group had been successfully cured of a bacterial infection after 11 days of treatment whereas none of the volunteers in the control group had been healed after day 11.

After the treatment, all the volunteers of the treatment group experienced a decline in gingival pain, a dry mouth feeling, gingival bleeding or sulcus bleeding, including signs of infection. In summary, the use of a periodontal paste dressing and a gingival brushing solution to supplement the SRP treatment is extremely beneficial for volunteers in comparison with those receiving only SRP treatment.

On the whole these results from example 9 show that the gingival paste dressing and the gingival brushing solution are effective and safe and tolerated well as a supplement to SRP in management of chronic periodontitis.

Although specific embodiments were described above, it is obvious that different combinations of the embodiments presented can be used as long as the options do not mutually preclude one another.

Whereas the invention was described above with reference to specific embodiments, it is obvious that changes, modifications, variations and combinations are possible without deviating from the idea according to the invention.

What is claimed is:

1. A method for treatment of periodontitis, gingivitis or gingival bleeding, comprising:
dissolving a self-dissolving tablet or powder in water, the self-dissolving tablet or powder comprising an active composition, wherein the active composition comprises at least one organic or inorganic chlorine compound and at least one oxidizing agent, the at least one oxidizing agent comprising at least one persulfate compound and the at least one organic or inorganic chlorine compound comprising a chloride compound in the form of a salt, the water and dissolved tablet or powder forming an aqueous solution; and
upon dissolution of the tablet or powder, applying the aqueous solution to teeth, gingiva, gingival margins or mucous membranes.

2. The method according to claim 1, wherein the method further comprises treating marginal periodontitis or as an adjuvant treatment for apical periodontitis, treating periodontitis in conjunction with a dental cleaning treatment, or treating periodontitis following a scaling and root planing treatment.

3. The method according to claim 1, wherein the active composition comprises a carboxylic acid comprising a monocarboxylic or dicarboxylic acid.

4. The method according to claim 3, wherein the monocarboxylic or dicarboxylic acid is contained in such an amount in the active composition that the active composition, dissolved in a certain amount in the aqueous solution, creates a pH of <6.

5. The method according to claim 1, wherein the active composition further comprises a hydrogen peroxosulfate compound comprising at least one potassium hydrogen monopersulfate (KHSO₅), as the at least one oxidizing agent.

6. The method according to claim 1, wherein the chloride compound in the form of a salt is selected from a group of alkali salts and alkaline earth salts.

7. The method according to claim 1, wherein an oxidation potential of the at least one oxidizing agent in the aqueous solution is higher than an oxidation potential of Cl¹⁻/Cl⁰.

8. The method according to claim 1, wherein the active composition comprises at least one substance for accelerating dissolving of a carbonate or bicarbonate-containing compound.

9. The method according to claim 1, wherein the active composition excludes fluoride salts.

10. A method of topical application of an active composition on teeth, gingiva, gingival margins or mucous membranes for treating periodontitis, gingivitis or gingival bleeding or inflammation, comprising:
adding a self-dissolving tablet or powder to water to dissolve the table or powder to form an aqueous solution, the tablet or powder comprising an active composition; and
after dissolution of the tablet or powder in the water, applying the aqueous solution to teeth, gingiva, gingival margins or mucous membranes;
wherein the active composition comprises at least one organic or inorganic chlorine compound and at least one oxidizing agent, the oxidizing agent selected from a group of persulfate compounds, and the at least one organic or inorganic chlorine compound selected from a group of chloride compounds in the form of a salt.

11. The method according to claim 10, further comprising, prior to applying the aqueous solution to teeth, gingiva, gingival margins or mucous membranes, applying a cream, a paste, an ointment or a gel to the teeth, the gingiva, the gingival margins, or the mucous membranes, wherein the cream, paste, ointment or gel comprises a combination or mixture of the active composition and an adhesive composition.

12. The method according to claim 11, further comprising applying the cream, paste, ointment or gel at least a day before application of the aqueous solution to teeth, gingiva, gingival margins or mucous membranes.

13. The method according to claim 12, further comprising repeating application of the aqueous solution to teeth, gingiva, gingival margins, or mucous membranes for several days.

14. The method according to claim 11, wherein the adhesive composition comprises at least one alcohol, in an amount of up to 10% by weight, based on a total amount of the adhesive composition.

15. The method according to claim 11, wherein the adhesive composition comprises at least one polysaccharide, in an amount of up to 20% by weight, based on a total amount of the adhesive composition.
